(19) United States
(12) Patent Application Publication
(10) Pub. No.: US 2014/0135284 A1
(43) Pub. Date: May 15, 2014

(54) COMPOSITION OF AQUEOUS BUFFER SOLUTION FOR THE TREATMENT OF CELLULAR ENVIRONMENT AND ION CHANNELS AND METHODS FOR USING SAME

(71) Applicant: Stephen H. MONROE, Portland, OR (US)

(72) Inventor: Stephen H. MONROE, Portland, OR (US)

(21) Appl. No.: 14/073,153

(22) Filed: Nov. 6, 2013

Related U.S. Application Data
(62) Division of application No. 12/747,890, filed on Jun. 11, 2010, now abandoned.
(60) Provisional application No. 61/007,228, filed on Dec. 11, 2007.

(51) Int. Cl. A61K 31/728; A61K 31/194; A61K 31/19; A61K 31/375

(52) U.S. Cl. CPC A61K 31/728; A61K 31/375; A61K 31/194; A61K 31/19
USPC 514/54; 514/474; 514/574; 514/557

(57) ABSTRACT

This invention relates to a buffering composition for the topical or internal treatment of animal tissues to buffer and normalize the pH of the cellular environment while also affecting specific cellular membrane ion channels as a method to affect inflammation, proteases, reactive oxygen species and free radicals, the composition containing a pharmaceutically-effective concentration of an acid and its base salt, where the counterions of the base salt are potassium and at least one of cesium and rubidium, or all three.

### CONTINUITY AND CLAIM OF PRIORITY

This is a divisional application of U.S. patent application Ser. No. 12/747,890 filed 11 Jun. 2010, which claims priority to U.S. provisional patent application No. 61/007,228 filed 11 Dec. 2007 and international patent application no. PCT/US2008/086477 filed 11 Dec. 2008.

### BACKGROUND OF THE INVENTION

This invention relates to a composition for the topical or internal treatment of animal tissues to buffer and normalize the pH of the cellular environment while also effecting specific cellular membrane ion channels as a method to affect inflammation, proteases, reactive oxygen species and free radicals.

### SUMMARY OF THE INVENTION

Many disease states or medical procedures result in a change in the pH balance of the cellular environment. This, in turn, causes the cells to begin producing abnormal types or amounts of metabolic products such as inflammatory cytokines, proteases, and various radical species. This is true in cancers as well as tissue low in oxygen or in a hypoxia state as well as a response to tissue disruption or surgical procedures. Stress also causes changes in cellular and extracellular pH levels.

The present invention seeks to treat and benefit cellular states and potential disease causing effects by buffering the cellular environment at specific levels combined with cellular membrane electric potential changing cations such as sodium or potassium or rubidium or cesium or a combination thereof serving as the buffer counter ion. These cations can be selected to affect specific cellular ion channels such as the sodium ion channel, the potassium ion channel or others depending on the ion selected. The buffer can be composed of any typical buffer such as ascorbic acid or citric acid or Tris or phosphate or acetate or similar buffers or more exotic acids or alkalis such as Hyaluronic acid depending on the pH of the buffered solution desired.

Additional features of the disclosure will become apparent to those skilled in the art upon consideration of the following detailed description of preferred embodiments exemplifying the best mode of carrying out the invention as presently perceived.

### DETAILED DESCRIPTION OF EMBODIMENTS OF THE INVENTION

The present invention relates to compositions for controlling the pH of the cellular environment and the cellular membrane ion channels in order to reduce and/or prevent the production of inflammatory cytokines, reactive oxygen species and proteases that can be upregulated by an abnormal pH environment, to pharmaceutical and cosmetic compositions containing them, and to their use in the treatments of ailments associated with inflammation, reactive oxygen species, and proteases for example, including but not limited, to skin cancers, cancers, inflammation, sunburns, wounds, arthritis, eye diseases, gum diseases, psoriasis, atopic dermatitis, Rosacea, or other diseases in which inflammation and tissue degradation are part.

This invention combines ingredients in a unique combination to achieve three different goals with the resultant outcome being a method for helping to control inflammation, tissue degradation, and the consequent degenerative cascade. In the first place, this unique combination is used to scavenge or otherwise reduce oxygen radicals, other free radicals, and Reactive Oxygen Species. In the second, this unique combination is used to prevent or reduce the inflammation of tissues and prevent or reduce the production of inflammatory cytokines. In the third place, this unique combination is used to prevent or reduce the production or expression of proteases.

Inflammation is a normal response to a variety of assaults and stresses on various tissues in mammals. Generally it is a beneficial response that allows the body to protect or repair itself. It is also very tightly controlled under normal circumstances with many redundant control mechanisms. When inflammation is out of control, as is typically found in many diseases, especially chronic diseases, it can do devastating damage to the tissues involved.

Once inflammation is out of control, it can be very difficult to regain homeostasis and until that point is reached, a great deal of damage may be done to the tissues involved. The inflammatory process itself is self reinforcing with many different cytokines and chemical species up-regulating the inflammatory response. Controlling this process through exogenous additives can be assisted by effecting specific cell receptors such as is clone with a variety of NSAIDS (Non Steroidal Anti-Inflammatory Drugs), corticosteroids, or others. This present invention takes a different approach by using a multi-variate approach to address some of the fundamental causes of inflammation and helping control the negative consequences of inflammation by reducing its stimulation.

The pH of tissue is normally very tightly controlled, but can be disturbed by tissue damage and other assaults on the tissues. Once the pH has been moved from this equilibrium condition, the cells in various tissues expresses a variety of responses that result in the generation of NO, ROS, free radicals and other chemical species. These in turn induce the upregulation of inflammatory cytokines ranging from TNF-a, IL-1, IL-2, IL-6, and others. These then up-regulate other cytokines as well as a variety of tissue degrading proteases.

As one aspect of the present invention, by controlling the pH of the tissue, it is possible to reduce the generation of the free radical species, NO, and ROS and others. This will help reduce the stimulation of inflammation in that tissue and its environs. As a second aspect of the present invention, by scavenging the free radicals that may exist with an appropriately chosen acid/base combination, it is possible to further reduce the inflammatory stimulus. Furthermore, as a third aspect of the present invention, evidence shows that changes in ion transport through cellular membrane ion channels does affect the production of inflammatory cytokines and can thus reduce inflammation. As a fourth aspect of the present invention, reducing inflammation will reduce the stimulated production of a wide variety of proteases which damage tissues, the fragments of which can induce the inflammatory cascade. As a fifth aspect of the present invention, literature indicates that the judicious selection of the ions used can reduce the levels of proteases expressed through the regulation of their mRNA. This current invention works by addressing these areas of inflammation up-regulation.

The buffer helps control the pH of the tissue environment. The addition of citric and other acids and of Rubidium helps scavenge free radicals and ROS. The addition of the salts of these acids affects the specific ion channels involved and taken together the effects independently and severally clown-regulate inflammation and the production of proteases.

The outcome of this treatment will be to help return the tissue to homeostasis and a normal state. This will apply to, but in no way is limited to, the following examples. In the case of cancers, this will help prevent tumors from growing and potentially shrink them. In the case of wounds, especially chronic wounds, this will allow the tissue to heal normally. In the case of Psoriasis, atopic dermatitis and other skin conditions, this will prevent the symptoms of those diseases. In the case of Arthritis, this will help prevent the damage to the joint. In the case of gingivitis and periodontal disease, this will help prevent the degradation of the gum tissue. In the case of Macular Degeneration, this will prevent angiogenesis and the proliferation of blood vessels. In the case of sunburns, this will help prevent to damage to the epidermis. In the case of skin aging, this will help prevent collagen damage and the consequent wrinkles.

These are just a few of the potential applications. The full range of applications will apply to any condition in which there is tissue degradation.

In the area of wound care, wounds typically become more acid than normal tissue. This pH change can induce a cascade of further events such as the recruitment of neutrophils which in turn can initiate an inflammatory cascade. This can begin with the generation of oxygen radicals and the production of other Reactive Oxygen Species (ROS) which leads to the upregulation of pro-inflammatory cytokines such as TNF-a, IL-1, IL-6, IL-8 and others. This in turn leads to further degradation of the tissue as proteases are induced. If this is not brought under control, it is possible that a normal acute wound can become a chronic, non-responding wound.

A recent study (Weindorf, M. et al, Zeitshrift fur Wund Heilung, 12 (2): 1-4, May 2007, incorporated herein by reference) has shown that the underlying pH of the major wound care dressings used in Europe are basically uncontrolled, ranging from a pH of 2.2 to a pH of 11.70. By not controlling the pH of the wound environment, these dressings can actually contribute to a wound's pathology by inducing further inflammation and tissue breakdown. Kellum, et al (Kellum, J. et al J Leukoc. Biol, 69: 522-530, 2001, incorporated herein by reference) has shown that a moderately reduced pH, even at a pH between 6-7 will upregulate inflammatory cytokines. A pH reduction from 7.4 to7.0 will upregulate NO (nitric oxide) which in turn upregulates the inflammatory cascade.

Kellum further found different effects depending on the acid species studied. Hydrochloric acid (HC1) upregulated inflammation even with small reductions in pH. Lactic acid, in contrast, was an effective anti-inflammatory.

Coakley et al. (Coakley, R. et al, Blood 100 (9):3383-91, 2002, incorporated herein by reference) found that a decrease in pH has a strong affect on neutrophils which can induce tissue necrosis. They further found that alkalizing the tissue environment can protect the neutrophils and help prevent necrosis. Gerwick et al. (Gerwick, L. et al, Cancer Res. 56: 1194-98, 1996, incorporated herein by reference) found that cancer tumors are in general more acidic than normal tissue. Xu et al. (Xu et al, Cancer Res. 60: 4610-16, 2000, incorporated herein by reference) found that an acidic pH in ovarian cancer cells produced elevated levels of the inflammatory cytokine IL-8.

Razaq (Razaq, S. et al, European Spine J. 12 (4):341-9, 2003, incorporated herein by reference) found that an acidic pH dramatically reduced the body's normal system for controlling the tissue degrading proteases. At an acidic pH, bovine disks showed that the Tissue Inhibitors of Metalloproteinases (TIMPs) by more than 50% in general and TIMP 1 by more than 90%. Since these inhibitors are a key mechanism for controlling MMPs, this significant reduction in TIMPs will result in the overexpression of MMPs and thus an excessive level of tissue degradation.

Qian Shi et al. (Qian Shi, et al, J. Interferon & Cytokine Res. 20 (11):1023-28, 2000, incorporated herein by reference) found that a mild acidosis of tumor pH to a pH of 6.4 resulted in increased levels of NF-KB. This increased IL-8, which in turn lead to an increase in inflammation and enhanced tumor progression.

Martin, et al. (Martin, P et al, Trends Cell Biol. 15: 599-607, 2005, incorporated herein by reference) found that the inflammatory response is not necessary for the tissue repair process. His work with PU-1 null mice shows that they will heal normally and with no fibrosis of the wounded tissue compared to normal mice. The PU-1 null mice lack neutrophils thus showing it is possible to still heal a wound without neutrophils that are normally upregulated by inflammation. Further work showed that inhibiting the inflammatory response can actually accelerate wound healing with a decrease in granulation tissue and scar formation.

Hayden (Hayden, M. et al, Cardiovascular Diabetology 1:3-30, 2002, incorporated herein by reference) noted that a pH decrease led to redox stress in the cells involved. This in turn resulted in a net increase in oxygen radicals, which in turn led to an increase in MMPs. He also noted that an increase in redox stress leads directly to an increase in ROS.

These observations demonstrate the importance of pH as a causative factor in stimulating a response from the tissues involved.

As Kellum et al. (Kellum, J. et al, Critical Care 8: 331-336, 2004, incorporated herein by reference) state,
Understanding the effects of acid-base balance on the inflammatory response is highly relevant to clinical medicine for a variety of reasons. First, current deficiencies in our understanding of the effects of acidosis on a wide range of cellular processes have led to controversy in the way in which patients are managed in a variety of clinical settings. Most clinicians tend to ignore the effects of exogenous CL and pH, but many will treat even mild forms of acidemia. . . . Second, our ability to alter acid-base balance as a tool with which to manipulate cellular processes will be dependent on an improved understanding of the relationship between pH and the synthesis and release of inflammatory molecules.

This clearly demonstrated the importance of controlling the pH of the tissue environment through the use of not just an acid or a base, but through the judicious use of buffers.

A second factor in this current invention is the use of specific counterions to induce beneficial results from the manipulation of the ion channels involved and manipulation of the cellular membrane potential.

Eisenhut (Eisenhut, M., J. of Inflammation 3 (5):1-15, 2006, incorporated herein by reference) reports that changes in ion transport does affect the expression of inflammatory cytokines. Hsiau et al. (Hsiau, T. Report—Research Science Institute, July 2003, incorporated herein by reference) found that potassium ion channel inhibition caused a disruption in tissues regeneration in planarium. Roger et al. (Roger, S. et al, Current Pharmaceutical Design 12 (28) 3681-95, 2006, incorporated herein by reference) shows that voltage gated sodium ion channels are involved in the metastasis of cancer cells.

Chacon et al. (Chacon Cruze, E. et al., J Leucocyte Biology, 64:759-66, 1998, incorporated herein by reference) report that membrane depolarization has an anti-inflammatory effect by preventing the calcium induced response of neutrophils.

Van den Berg et al. (Van den Berg, A, et al, J. Wound Care 12 (10): 1-5, 2003, incorporated herein by reference) report that metal ions can inhibit complement activation and TOS production by Polymorphonuclear neutrophils (PMNs).

Hanley et al. (Hanley, P. et al., PNAS 101 (25):9479-84, 2004, incorporated herein by reference) show that IL-6 can be induced by membrane depolarization. This can be either pro- or anti-inflammatory. Calcium induces inward rectifying potassium ion channels which induces depolarization of the membrane which in turn induces up to a 40 fold increase in IL-6.

### Inflammation and Matrix MetalloProteinases (MMPs)

As one of the most important inflammatory cytokines, TNF-a, has long been recognized to increase MMPs. This leads to the proteolytic degradation of tissues, the fragments of which lead to the further expression of inflammatory cytokines in a self-reinforcing loop.

Monroe et al. (Monroe, S. et al, poster presentation, AAWC, 2004, incorporated herein by reference) showed that four metal ions in combination, potassium, rubidium, calcium and zinc (K, Rb, Ca, and Zn) reduced the genetic expression of a wide group of MMPs.

This current invention teaches how the combination of controlling pH through utilization of buffers, impacting specific ion channels through the use of counterions selected from sodium, potassium, rubidium, or cesium, and reducing the effect of free radicals and other Reactive Oxygen Species (ROS) is used to positively effect inflammation and the expression of proteases.

One embodiment of this invention is the utilization of citric acid and potassium citrate to create a buffer with an effective pH between 2.0 and 7.0. This can be incorporated into an aqueous solution, an ointment, or as a powder. Also with a concentration up to 1000 mmolar.

A second embodiment is the utilization of citric acid and rubidium citrate to create a buffer with an effective pH between 2.0 and 7.0. This can be incorporated into an aqueous solution, an ointment, or as a powder. Also with a concentration up to 1000 mmolar.

A third embodiment is the utilization of citric acid and sodium citrate to create a buffer with an effective pH between 2.0 and 7.0. This can be incorporated into an aqueous solution, an ointment, or as a powder. Also with a concentration up to 1000 mmolar.

A fourth embodiment is the utilization of citric acid and cesium citrate to create a buffer with an effective pH between 2.0 and 7.0. This can be incorporated into an aqueous solution, an ointment, or as a powder. Also with a concentration up to 1000 mmolar.

A fifth embodiment is the utilization of citric acid and potassium citrate combined with rubidium citrate to create a buffer with an effective pH between 2.0 and 7.0. This can be incorporated into an aqueous solution, an ointment, or as a powder. Also with a concentration up to 1000 mmolar

Another embodiment is the utilization of lactic acid and potassium lactate citrate to create a buffer with an effective pH between 2.0 and 7.0. This can be incorporated into an aqueous solution, an ointment, or as a powder. Also with a concentration up to 1000 mmolar.

A further embodiment combines different buffers in combination to achieve specific effects. For example, citric acid is an effective ROS scavenger. Hyaluronic acid helps heal damaged tissues and more specifically has been used to help reduce adhesions typical after abdominal surgery. A combination of citric acid and Hyaluronic acid and their buffers would be expected to combine the beneficial effects of both buffer species.

While the disclosure is susceptible to various modifications and alternative forms, specific exemplary embodiments thereof have been shown by way of example and have herein been described in detail. It should be understood, however, that there is no intent to limit the disclosure to the particular forms disclosed, but on the contrary, the intention is to cover all modifications, equivalents, and alternatives falling within the spirit and scope of the disclosure as defined by the appended claims.

A plurality of advantages arises from the various features of the present disclosure. It will be noted that alternative embodiments of various components of the disclosure may not include all of the features described yet still benefit from at least some of the advantages of such features. Those of ordinary skill in the art may readily devise their own implementations that incorporate one or more of the features of the present disclosure and fall within the spirit and scope of the disclosure and claims.

I claim:

1. A method comprising:
applying a buffering composition containing a pharmaceutically-effective concentration of an acid and a complementary base salt of the acid, counterions of the complementary base salt including potassium and at least one of cesium and rubidium, to an area of a body exhibiting inflammation; and
monitoring an effect of the buffering composition on the area of the body to adjust a subsequent application of the buffering composition.

2. The method of claim 1, wherein the counterions of the complementary base salt include all of potassium, cesium and rubidium.

3. The method of claim 1, wherein the inflammation is associated with psoriasis.

4. The method of claim 1, wherein the inflammation is associated with rosacea.

5. The method of claim 1, wherein the pharmaceutically-effective concentration of the acid and the complementary base salt is less than 1,000 mmolar.

6. The method of claim 1, wherein the acid is ascorbic acid.

7. The method of claim 1, wherein the acid is citric acid.

8. The method of claim 1, wherein the acid is hyaluronic acid.

9. The method of claim 1, wherein the acid is lactic acid.

10. The method of claim 1, wherein the acid is a mixture of citric and hyaluronic acids.
